(19) United States
(12) Patent Application Publication
(10) Pub. No.: US 2013/0041166 A1
(43) Pub. Date: Feb. 14, 2013

(54) PROCESS FOR THE PREPARATION OF DIENOGEST SUBSTANTIALLY FREE OF IMPURITIES

(71) Applicants: Suhas Ganpat Tambe, Pune (IN); Bhushan Bhanudas Kirange, Pune (IN); Gurvinder Pal Singh, Pune (IN); Purna Chandra Ray, Pune (IN)

(72) Inventors: Suhas Ganpat Tambe, Pune (IN); Bhushan Bhanudas Kirange, Pune (IN); Gurvinder Pal Singh, Pune (IN); Purna Chandra Ray, Pune (IN)

(73) Assignee: LUPIN LIMITED, Mumbai, Maharashtra (IN)

(21) Appl. No.: 13/642,390

(22) PCT Filed: Apr. 13, 2011
(86) PCT No.: PCT/IB2011/000825

§ 371 Date: Oct. 19, 2012

(30) Foreign Application Priority Data
Apr. 20, 2010 (IN) 438/KOL/2010

(51) Int. Cl. C07J 41/00

(52) U.S. Cl. 552/648

(57) ABSTRACT

The present invention provides novel process for preparation and purification of dienogest (I). The present invention provides dienogest (I) substantially free of impurities.

### FIELD OF THE INVENTION

The present invention is related to dienogest that is substantially free of impurities and process for its preparation.

### BACKGROUND OF THE INVENTION

Dienogest is an orally active synthetic progesterone (or progestin), used as an oral contraceptive in combination of ethinylestradiol. It has antiandrogenic activity that can improve androgenic symptoms. It is a non-ethinylated progestin which is structurally related to testosterone.

Dienogest is chemically known as, (17α)-17-Hydroxy-3-oxo-19-norpregna-4,9-diene-21-nitrile or 17α-Cyanomethyl-17β-hydroxy-4,9-estradien-3-one and it is represented by following structure:

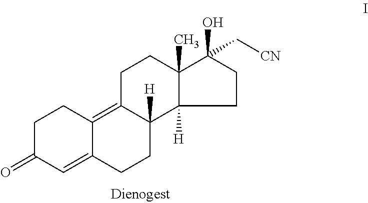

The U.S. Pat. No. 4,248,790, describe process for preparation of dienogest as in Scheme-I.

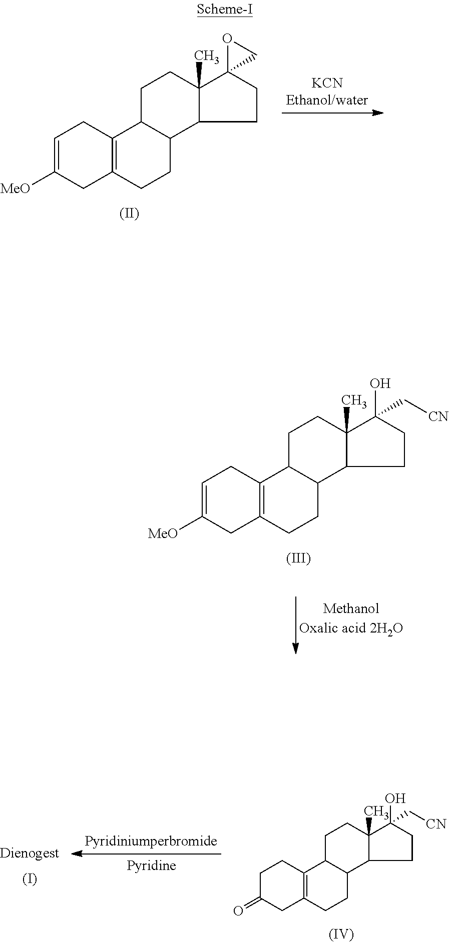

The publication Zhang Xiuping et al., Pharmaceutical Industry (1985), 16(9), 399-401, describe the conversion of ketal (3,3-dimethoxy-estra-5(10),9(11)-diene-17-one) (V) to dienogest (I) in three steps as shown in Scheme-II, wherein ethylene oxide intermediate (VI) is reacted with alcohol solution of potassium cyanide to produce (VII) followed by acid hydrolysis.

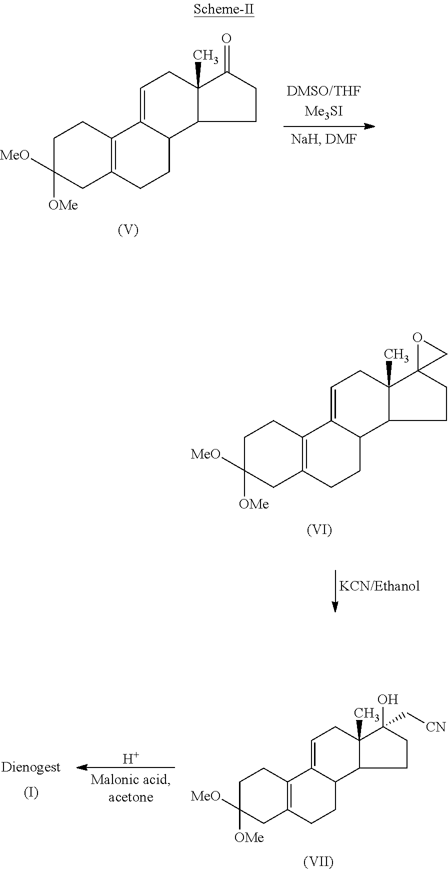

The patent EP 1935898 disclose preparation of dienogest (I) in which the ketal (V) is reacted with CNCH₂CeCl₂, followed by treatment with acetic acid and hydrochloric acid.

The German patent application DD 296,495 describes a one-pot synthesis of dienogest from 3,3-dimethoxy-estra-5(10),9(11)-diene-17-one (V), on treatment with dilute sulfuric acid.

Prior art processes described above use toxic reagents like trimethyl sulfonium iodide or alkali cyanides. Removal of residual cyanide demands additional operations of destruction of cyanide which makes it a costly method.

Several methods are known in the literature for purification of dienogest by crystallization. These are discussed below.

The U.S. Pat. No. 4,248,790, discloses crystallization of dienogest from ethyl acetate and 80% acetonitrile. The publication Zhang Xiuping et al., Pharmaceutical Industry (1985), 16(9), 399-401, and patent DD 205170 disclose crystallization of dienogest from methanol. EP 1935898 and U.S. Pat. No. 5,955,622 disclose crystallization of dienogest from acetone.

The patent US 20080287404 A1 covers recrystallisation of dienogest from ethyl acetate, acetone, tert-butyl methyl ether, diisopropyl ether, acetonitrile, methanol, ethanol or aqueous mixtures of different ratio of these solvents.

The patent EP 1963354 B1 discloses purification of crude dienogest by preparative HPLC to obtain pure dienogest with total amount of impurities up to maximum 0.1% and individual impurities up to maximum 0.02% (by HPLC). This method has the disadvantage that it is difficult on production levels.

The U.S. Pat. No. 5,438,134 and EP 231671 discloses compound 17α-cyanomethyl-17β-hydroxy-13β-methyl-gona-5(10),9(11)-diene-3-one, which is referred herein as diene impurity.

The diene impurity is also mentioned as 19-Norpregna-5(10),9(11)-diene-21-nitrile, 17-hydroxy-3-oxo-, (17α).

It is always desirable to prepare pharmaceutical products of high purity having a minimum amount of impurities, in order to reduce adverse side effects and to improve the shelf life of active ingredient, as well as its formulation. In some cases, it has been found that high purity also facilitates in formulation process.

The present invention is directed to provide an improved process for the preparation of dienogest having minimum amount of impurities.

### OBJECTIVE OF THE INVENTION

The objective of present invention is to provide dienogest that is substantially free of impurities and a novel process for its preparation.

Another objective of the present invention is to provide novel method of purification of dienogest by crystallization from mixture of dimethylformamide and water.

### SUMMARY OF THE INVENTION

The present invention provides dienogest containing diene impurity, (17α-cyanomethyl-17β-hydroxy-5,9-estradien-3-one) less than 0.05% and other total impurities less than about 0.1% (area percentage by HPLC analysis).

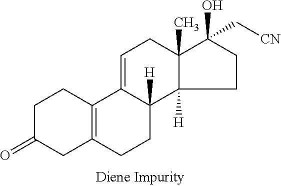

The present invention also relates to a novel process for preparing dienogest with a minimum amount of impurities.

The present invention further provides novel process of preparing dienogest which involves deprotection of ketal 3,3-dimethoxy-17α-cyanomethyl-17β-hydroxy-estra-5(10),9(11)-diene (VII) with perchloric acid.

A novel purification method by crystallization of dienogest from dimethylformamide-water mixture is also described.

### DETAILED DESCRIPTION

The present invention provides dienogest substantially free of impurities. In particular, the present invention provides dienogest having diene impurity less than 0.05% and other total impurities less than 0.1%, (measured as area percentage by HPLC).

The present invention further relates to a novel process for preparing dienogest which is substantially free of its impurities.

The conversion of ketal, 3,3-dimethoxy-17α-cyanomethyl-17β-hydroxy-estra-5(10),9(11)-diene (VII) by treatment with hydrochloric acid, sulfuric acid, acetic acid or oxalic acid to obtain dienogest is already known in the literature. However when these acids were used, it was observed that, the shift of the double bonds into conjugation with 3-keto group was incomplete. This resulted in the formation of dienogest (I) along with diene impurity in various amounts depending on the acid employed, which was difficult to remove by purification.

In order to control formation of diene impurity, a detailed investigation of reaction of ketal (VII) with various strong and weak acids was carried out. The observations of this study are provided in Table-1.

When strong acids such as hydrobromic acid, triflic acid, p-toluene sulphonic acid and trifluoroacetic acid were used, same difficulty was encountered; the shifting of the double bonds into conjugation with 3-keto group did not go to completion, resulting in the formation of diene impurity.

The use of weaker acids like oxalic acid led to very less shifting of double bonds and eventually gave diene impurity, as major product.

TABLE 1
Study of deprotection of ketal (VII) by using different acids at 20-30° C.

| S. | | | Dienogest (I) | Diene impurity |
|---|---|---|---|---|
| No. | Acid | Solvent | (%)^{#} | (%)^{#} |
| 1 | Conc. HCl | Acetonitrile | 91.13 | 8.1 |
| 2 | Hydrobromic acid | Acetonitrile | 78.05 | 19.43 |
| 3 | 30% sulfuric acid | Acetonitrile | 74.19 | 25.55 |
| 4 | p-Toluene sulfonic | Acetonitrile | 91.97 | 6.43 |
| | acid•H₂O | | | |
| 5 | Trifluoroacetic acid | Acetonitrile | * | * |
| 6 | Triflic acid | Acetonitrile | 89.32 | 5.4 |
| 7 | Oxalic acid•2H₂O | Methanol | 0.07 | 98.01 |
| 8 | Perchloric acid (70%) | Acetic acid | 93.75 | 2.84 |
| 9 | Perchloric acid (70%) | Ethyl acetate | 97.46 | 1.5 |
| 10 | Perchloric acid (70%) | Acetonitrile | 99.49 | 0.15 |
| 11 | Perchloric acid (70%) | Acetonitrile | 99.29 | 0.12 |

^{#}area percentage by HPLC
*degradation observed in TLC, HPLC data was not generated

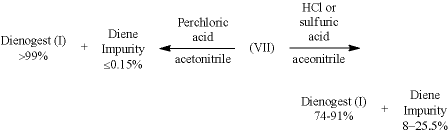

Conversion of ketal (VII) to dienogest, with perchloric acid was studied in different solvents such as acetic acid, ethyl acetate and acetonitrile. It was surprisingly found that, with perchloric acid, deprotection followed by shift of double bonds into conjugation with 3-keto group was almost complete to yield dienogest with minimum impurities (see entry 8-11 in Table-1). In acetic acid and ethyl acetate the diene impurity was slightly higher than in acetonitrile as solvent. Thus, the inventors found that perchloric acid in acetonitrile as solvent was the most suitable for conversion of ketal (VII) to dienogest (I) which provided dienogest of purity up to 99.49% and diene impurity less than or equal to 0.15%.

The conversion of ketal (VII) to dienogest with perchloric acid can be carried out at temperature ranging from 10 to 50° C., preferably 20-30° C.

Other solvents like propyl acetate, butyl acetate, ethyl propionate, propionic acid, propiononitrile, butyronitrile, dimethyl acetamide, dimethyl formamide, tetrahydrofuran, dioxane, acetone, methyl isobutyl ketone, methylethyl ketone; and mixtures thereof are also useful for conversion of ketal (VII) to dienogest, with perchloric acid.

In another aspect, the present invention provides novel method for purification of dienogest by crystallization from dimethylformamide (DMF)-water mixture. The purification afforded dienogest wherein the level of diene impurity was up to 0.02% and other total impurities less than 0.1%.

The advantage of the current invention is that the process is environment friendly as it does not use toxic reagents like trimethyl sulfonium iodide or alkali cyanides.

The dienogest prepared by the process of present invention is preferred for pharmaceutical formulation, since it is substantially free of impurities.

It will be apparent to those skilled in the art that various modifications and variations can be made in the present invention and specific examples provided herein, without deviating from the scope of the invention. Therefore, it is intended that the scope of the present invention covers the modifications and/or variations that are equivalents.

### SPECIFIC EXAMPLES

The present invention can be illustrated in one of its embodiments by the following non-limiting examples.

The purity of dienogest was measured as area percentage by HPLC with Zorbax Eclipse XDB, C-8 (4.6×150 mm), 5 μm as column, Water:Acetonitrile (90:10) and (10:90) as mobile phases at flow rate 1.0 mL/min, and UV detection at 240 nm.

The compound 3,3-dimethoxy-estra-5(10),9(11)-diene-17-one (V) was prepared from estra-4,6-diene-3,17-dione as described in the publication “Menzenbach, B et al., Pharmazie (1984), 39(7), 496-7”.

### Example 1

### 3,3-dimethoxy-17α-cyanomethyl-17β-hydroxy-estra-5(10),9(11)-diene

A mixture of n-hexyl lithium (33% solution in hexane), (180 g) and tetrahydrofuran (300 mL) was cooled to −50 to −40° C. A mixture of acetonitrile (33 mL) and tetrahydrofuran (200 mL) was added to it. A solution of 3,3-dimethoxy-estra-5(10), 9(11)-diene-17-one (V) (100 g, 0.32 mole) in tetrahydrofuran (600 mL) was added to the mixture at −50 to −40° C. and stirred for 90 minutes. After completion of reaction, temperature raised to −10 to 0° C. and water was added. Organic layer was separated, washed with brine and concentrated under vacuum. Residue was taken in acetonitrile, cooled to 0-5° C., stirred for one hour, filtered and dried under reduced pressure at 35-40° C. to give 100 g of title compound.

### Example 2

### Preparation of Crude Dienogest

The compound (100 g) of example-1 in acetonitrile (600 mL) was cooled to 0-5° C. and 70% perchloric acid (150 mL) was added and stirred for one hour at 20-30° C. After completion of reaction, water (1500 mL) was added and solid was filtered to obtain crude product. (HPLC data: Dienogest—99.55%, diene impurity—0.125%)

### Example 3

### Purification of Crude Dienogest

a) Wet solid from example-2 was charcoalised in dimethylformamide (400 mL) at 45-50° C. for one hour, filtered, washed with dimethylformamide (100 ml) and to the combined filtrate water (200 ml) was added. Reaction mass stirred for 2 hours at 0-5° C. Solid was filtered and dried under reduced pressure at 40-45° C. to give 66 g crude dienogest. (HPLC data: Dienogest—99.79%, Diene impurity—0.02%).
b) Dienogest (25 g) from example 3(a) was taken in dimethylformamide (125 mL) and heated to 40-50° C., filtered, washed with dimethylformamide (25 ml) and to the combined filtrate water (37.5 ml) was added to precipitate the product, cooled to 0 to 5° C. and stirred for 2 hours. Solid obtained was filtered, washed with water and dried under vacuum at 40-45° C. to give 23 g of Dienogest.(HPLC data: Dienogest—99.9%, Diene impurity—0.02%).

1. Process for preparation of dienogest of formula (I) 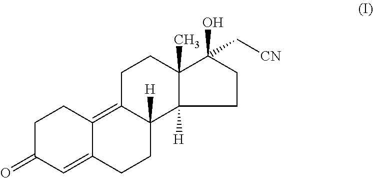
comprising reaction of 3,3-dimethoxy-17α-cyanomethyl-17β-hydroxy-estra-5(10),9(11)-diene (VII) 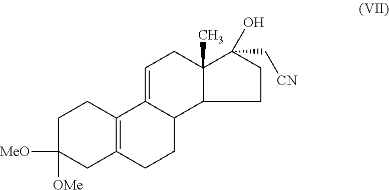 [SMILES: COC1(OC)CCC2=C(CCC3C2=CC[C@@]2(C)C3CC[C@@]2(O)CC#N)C1]
with perchloric acid.

2. The process according to claim 1, wherein the reaction can be carried out in presence of solvent selected from acetonitrile, acetic acid, ethyl acetate, propyl acetate, butyl acetate, ethyl propionate, propionic acid, propiononitrile, butyronitrile, dimethyl acetamide, dimethyl formamide, tetrahydrofuran, dioxane, acetone, methyl isobutyl ketone, methylethyl ketone and mixtures thereof.

3. The process according to claim 2, wherein suitable solvent is acetonitrile.

4. The process according to claim 1, wherein dienogest has the diene impurity 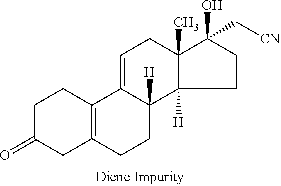
less than or equal to 0.15% and total impurities less than or equal to 0.5% measured as area percentage by HPLC.

5. Process for purification of dienogest (I) by crystallization from mixture of dimethylformamide and water.

6. The process according to claim 5, wherein dienogest has total impurities less than or equal to 0.25% measured as area percentage by HPLC.

7. The process according to claim 5, wherein dienogest has total impurities less than or equal to 0.1% measured as area percentage by HPLC.

8. The process according to claim 5, wherein dienogest has diene impurity less than or equal to 0.05% measured as area percentage by HPLC.

9. The process according to claim 5, wherein dienogest has diene impurity less than or equal to 0.02% measured as area percentage by HPLC.
